(19) United States
(12) Patent Application Publication
(10) Pub. No.: US 2011/0306873 A1
(43) Pub. Date: Dec. 15, 2011

(54) SYSTEM FOR PERFORMING HIGHLY ACCURATE SURGERY

(76) Inventors: Krishna Shenai, Toledo, OH (US); Ashok Biyani, Sylvania, OH (US); Vijay K. Goel, Holland, OH (US); Vijay Devabhaktuni, Toledo, OH (US); Bradford R. Lilly, Havre De Grace, MD (US)

(21) Appl. No.: 13/102,153

(22) Filed: May 6, 2011

Related U.S. Application Data
(60) Provisional application No. 61/332,290, filed on May 7, 2010.

(51) Int. Cl. A61B 5/05

(52) U.S. Cl. 600/424

(57) ABSTRACT

Methods and apparatuses for performing highly accurate surgery using a finite element model coupled with ultrasonic tracking are described.

### CROSS-REFERENCE TO RELATED APPLICATIONS

This application claims the benefit of U.S. Provisional Application Ser. No. 61/332,290 filed May 7, 2010, the entire disclosure of which is expressly incorporated herein by reference.

### STATEMENT REGARDING FEDERALLY SPONSORED RESEARCH

This invention was note made with any government support and the government no rights in the invention.

### TECHNICAL FIELD AND INDUSTRIAL APPLICABILITY OF THE INVENTION

The present invention relates to a system for performing surgeries, such as minimally invasive spinal fusion surgery. The system decreases overall radiation exposure, both to the surgeon as well as the patient. The system allows for an increase in overall accuracy of the surgical procedures, thus increasing success rates while also decreasing overall time for the surgery.

### BACKGROUND

After cancer and heart diseases, spinal disorders are the most expensive medical disorders in western countries. Low back pain significantly impacts health-related quality-of-life, increases use of healthcare resources, and results in increased socio-economic costs Treatments for spine disorders have produced a worldwide market for several years, which is expected to continue through the next decade.

There is a shift from traditional open spinal surgery to minimally invasive spinal surgery (MISS) with striking advantages in terms of scar size, muscle dilation vs. stripping, reduced bleeding, shortened hospitalization, and recovery time. The open spinal surgery leaves behind a large/long scar whereas the MISS procedure results in scars usually about an inch and a half.

For most navigational surgeries the patient gets a preoperative CT scan. The scan is then fed into the navigation system, which uses this data to give the surgeon anatomic landmarks. The patient positioning during surgery may change spinal alignment, and thus, the procedure may require extra visit for the patient to come to the hospital to have additional CT scans done. While the newer machines have fluoro-CT capability which can be performed once the patient is under anesthesia and positioned on the operating table prior to the surgical operation, there is a problem that fluoro-CT machines are large and are often in the surgeon's way during surgery.

Currently, minimally invasive surgeries, including spinal fusion, require exposure to high levels of radiation. Because the incision is small, the work area is not exposed. To compensate for this, many numbers of x-rays or images of other formats must be taken during the surgery to orient the surgeon. As this type of surgery grows in popularity, the surgeons performing them are exposed to increasing, and alarming, levels of radiation. Through the use of robotics, image analysis, and sensor based locationing systems it is possible to decrease these levels of exposed radiation, as well as increase accuracy and decrease operation time.

A typical spinal surgery (e.g., transforaminal lumbar interbody fusion) exposes surgical operating room (OR) personnel (surgeon, patient, and surgeon's assistants) to repetitive exposure of x-ray radiation (e.g., in the case of fluoroscopy for 2 to 4 minutes). Specifically, the surgeon's hand holding the mechanical device/screw is subject to excessive radiation exposures, and research toward minimizing such exposure is of paramount importance.

As there is progress from open surgeries to minimally invasive procedures, there is a need to improve techniques and increase safety both the surgical team and the patient.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In one aspect, the present invention generally relates to minimally invasive surgery and in particular, to a system for three-dimensional (3-D) tool tracking by using a tracking system to generate, derive and update data and move tools in response to such data (e.g., tool position, velocity, applied force and the like) during a minimally invasive robotic surgical procedure.

There is provided herein a tool tracking system which includes tracking a robotic tool by processing tool-state information using ultrasound coupled with a finite element (FE) 3-D model.

In certain embodiments, the tool-state information can be continuously provided at a sampling rate for processing. The tool-state information is a real-time updatable 3-D model which can be used to update the position of the tool while also estimating the state of the tool. This tool-state 3-D model information is generated from sensor data indicative of at least a position of the tool in a fixed reference frame. In certain embodiments, the sensor data can be provided by position sensors coupled to a mechanism for manipulating the tool through the incision in the body, and the tool-state 3-D model information is generated using the sensor data.

The sensor data can be provided by detecting a signal indicative of the position of the tool in a fixed reference frame. The signal can emanate from the tool and/or can be reflected off of the tool. Also, in certain embodiments, the tool state information can originate from ultrasound device.

The system described herein processes the tool-state information by generally generating a computer model of the tool that is positioned and oriented within an image plane defined by the initially gathered 3-D model data. The position and orientation of the computer model is modified with respect to an image of the tool in the image plane until the computer model approximately overlays the image of the tool so as to generate a corrected position and orientation of the tool.

For example, the system can include: receiving sensor information indicative of a position and orientation of a tool when the tool is inserted through an incision in a body; receiving ultrasound information for the tool; and determining the position and orientation of the tool using the ultrasound information.

The determination of the tool position and orientation can include one or more of: determining one or more estimated positions and orientations of the tool relative to a fixed reference frame from the sensor information; determining one or more estimated positions and orientations of the tool relative to an ultrasound reference frame from the image information; translating the one or more estimated positions and orientations of the tool from the fixed reference frame to the ultrasound reference frame; and processing the one or more estimated positions and orientations to generate the tool position and orientation relative to the ultrasound device reference frame.

The estimated positions/orientations derived from time sampled information can be provided by one or more sensors coupled to a mechanism for manipulating the tool through the incision in the body. The ultrasound estimated positions/orientations can be derived from sampled ultrasounds provided by one or more ultrasound devices.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1: A schematic diagram of a minimally invasive robotic surgery system.

FIG. 2: A simplified schematic diagram of a minimally invasive robotic surgery system.

FIG. 3: Example of a 3D model that can be generated and then uploaded into software to plot a surgery.

FIG. 4: Schematic illustration of a tracking system.

FIG. 5 and FIG. 6 illustrate 3D FE models generated using ABAQUS software.

FIG. 7: A model of the, indicating where loads were applied and measured. Black arrow indicates load point, white arrow indicates point where measured.

FIG. 8A: A schematic diagram of a minimally invasive robotic surgery system setup.

FIG. 9, FIG. 10 and FIG. 11 show the measurement results from the displacements applied in the test directions on the spine analog:

FIG. 9: Graph showing 3 cm in −X applied at base, measured at 3^{rd} vertebrae from base.

FIG. 10: Graph showing 3 cm in −X applied at base, measured at 3^{rd} vertebrae from base.

FIG. 11: Graph showing 3 cm in −X applied at base, measured at 3rd vertebrae from base.

FIG. 12: Table showing standard deviations in centimeters.

FIG. 13: A graph showing solve times in seconds vs. # of threads (single machine) directions. The Y direction would have been a compression test which is

FIG. 14: Graph showing solve time in seconds vs. # of processes (MPI) directions.

FIG. 15: Schematic illustration of System Architecture Layout

FIGS. 16A-16C: Photographs of test setups. ENDPOINT designated by large tracker. MIDPOINT designated by nail head.

FIG. 17: Tables 1-4 showing deltas between tracked and calculated data.

FIG. 18: Photograph showing robot final position.

FIG. 19A: CT scan of the thoracic region.

FIG. 19B: ABACUS FEM model of lumbar spine.

FIG. 20: Steps in the development of 3D FE model of lumbar spine segment using CT scans.

FIG. 21: A schematic diagram of a wireless interface capable of two-way communication with a large number of sensor nodes.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

The present invention is an improvement in the currently developing medical and surgical field where a surgeon at a central workstation perform operations remotely on a patient who had been pre-operatively prepared by a local surgical and nursing personnel. This surgical telepresence permits expert surgeons to operate from anywhere in the world, thus increasing availability of expert medical care. This new “haptic” technology will enable the surgeon to have tactile and resistance feedback as she operates a robotic device. With the minimally invasive surgical system described herein, the surgeon is able, via a robotic system to surgically place screws and/or pins in a bone, to feel the resistance of the screw/pin against the bone as she would if working directly on the patient.

In one embodiment, the system for conducting minimally invasive surgery, includes:

a three-dimensional (3-D) finite element (FE) model of a surgical working area that can be updatable in substantially real-time;

one or more markers operable to be placed at the surgical working area in at predetermined locations, the trackers being operable to register locations of the markers at identical locations in the 3-D model;

a robotic system operable to know the exact location and position of surgical working area; and

a software program operable to: i) track the location of the markers as the surgical working area is being deformed and/or displaced by action of the robotic system; and ii) update the 3-D model so that the robot can be guided to perform one or more tasks at the surgical working area without any substantial time delay.

In certain embodiments, the software program is operable to compute the displacements/deformations that are likely to occur due to the force applied the actions of the robotic system.

In another embodiment, a system for conducting minimally invasive surgery includes:

i) obtaining a three-dimensional (3-D) finite element (FE) computer model of a surgical working area on a patient;

ii) determining at least one of a position and orientation of a surgical tool that is positioned and oriented within an image plane defined by the 3-D model; and

iii) modifying at least one of the position and orientation of the 3-D model with respect to the image of the tool in the image plane such that the 3-D model approximately overlays the image of the tool so as to generate a corrected position and orientation of the tool; and

iv) tracking the tool by processing tool state information from step iii) using ultrasound coupled with the 3-D model.

In certain embodiments, the tool state information is continuously provided at a sampling rate for processing.

In certain embodiments, the signal emanates from the tool.

In certain embodiments, the signal reflects off of the tool.

In certain embodiments, the determination of the tool position and orientation comprises:

determining one or more estimated positions and orientations of the tool relative to a fixed reference frame from the sensor information;

determining one or more estimated positions and orientations of the tool relative to an ultrasound reference frame from the image information; translating the one or more estimated positions and orientations of the tool from the fixed reference frame to the ultrasound reference frame; and

processing the one or more estimated positions and orientations to generate the tool position and orientation relative to the ultrasound reference frame.

In certain embodiments, the one or more estimated positions and orientations derive from time sampled information provided by one or more sensors coupled to a mechanism for manipulating the tool through the incision in the body, and the one or more ultrasound estimated positions and orientations derive from sampled ultrasounds provided by one or more ultrasound devices so as to capture locations of the tool.

In certain embodiments, one or more measures are derived for the one or more estimated positions and orientations. Further, in certain embodiments, the measure for the one or more estimated positions and orientations is determined from a difference between one of the estimated positions and a position being commanded by a command signal controlling the mechanism for manipulating the tool.

In certain embodiments, the determination of the tool position and orientation includes processing the ultrasound information to identify a marker on the tool, and determine an orientation of the tool using the marker.

In certain embodiments, the determination of the tool position and orientation includes:

generating a computer model of the tool using the ultrasound sensor information so as to be positioned and oriented within a plane defined in the ultrasound information, and

modifying the position and orientation of the computer model with respect to an image of the tool in the image plane until the computer model substantially overlays the image.

In another aspect, there is provided herein a minimally invasive robotic surgery system, comprising:

one or more ultrasound devices operable to provide data from which tool state information is generated when a tool is inserted and robotically manipulated through an incision in a body; and

a processor operable to process the non-endoscopically and endoscopically derived tool state information for tracking the state of the tool.

In certain embodiments, the system can further comprise a mechanism used for manipulating the tool through the incision in the body, wherein the one or more ultrasound devices include one or more sensors providing sensor data representing tool movement information according to such manipulation.

The sensor data can include digitized samples of an identifiable signal emanating from or reflecting off the tool so as to indicate the position of the tool.

Further, the processor can be further operable to identify a marker on the tool, and to determine an orientation of the tool using the marker while tracking the state of the tool.

The system can include a mechanism used for manipulating the tool through the incision in the body, wherein the sensor data represents kinematic information according to such manipulation.

The processor can be operable to generate a 3-D computer model of the tool positioned and oriented within an image plane defined in the ultrasound captured data, and modify the position and orientation of the 3-D computer model with respect to an image of the tool in the image plane until the 3-D computer model substantially overlaps the image.

The modification of the estimated position and orientation of the 3-D computer model with respect to the ultrasonic data of the tool in the captured image, can include determining the modified position and orientation of the computer model that approximately overlays the tool image by minimizing a difference between the computer model and the ultrasonic data of the tool.

In yet another aspect, there is provided herein a tool tracking method comprising:

generating a plurality of estimated tool states for each point in a plurality of points in time, while the tool is inserted and being manipulated through an incision in a body; and

determining an optimal estimated tool state for each point in the plurality of points in time by processing the plurality of estimated tool states using ultrasonic techniques.

wherein the plurality of estimated tool states include an estimated tool state determined using only sensor data associated with a robotic mechanism for manipulating the tool, so as to be indicative of movement of the robotic mechanism.

In certain embodiments, the method can include wherein the plurality of estimated tool states includes an estimated tool state determined using only sensor data associated with the tool, so as to be indicative of a position of the tool.

Further, the plurality of estimated tool states can include an estimated tool state determined using only ultrasound data generated by an external ultrasound device positioned so as to detect a tool inserted into and being manipulated through a incision in the body.

In still another aspect, there is provided herein a minimally invasive surgical robotic system, comprising:

a tracking system for a robotic system operable to send signals;

a computer interface operable to receive the sent signals from the tracking system and to combine the sent signals with a three-dimensional (3-D) finite element (FE) computer model to provide sensor data;

the computer interface operable to transmit the sensor data to the robotic system; and

the computer interface operable to provide a closed loop system operable to transmit/receive sensing and feedback signals from the tracking system as a surgery is being performed;

wherein a real-time computer modeling is provided during surgery; the real-time computer modeling comprising an updatable three-dimensional (3D) finite element (FE) modeling of a surgical work area as such surgical work area is being displaced or deformed by the robotic action.

The minimally invasive surgical robotic system can be operable to navigate using precise control signals wirelessly transmitted from a control station.

In certain embodiments, the minimally invasive surgical robot arm contains end-effectors and sensors that provide appropriate feedback signals

In certain embodiments, wherein the surgery being performed is any spinal surgical procedure including drilling, screwing and implant insertion.

In certain embodiments, the tracking system includes one or more reference points embedded at or near on the surgical working area and which appear in the three-dimensional (3D) finite element (FE) model of the surgery surgical working area.

In certain embodiments, as the surgical working area is displaced and/or deformed due to the robotic action, the tracking system interfaces with the computer to generate a real-time update of the 3D FE model corresponding to the new position and shape of the object.

In certain embodiments, the surgical working area is a patient's spine, and the three-dimensional (3D) finite element (FE) modeling of the patient's spine contains trackers placed at MIDPOINT (MP) nodes and ENDPOINT (EP) nodes in the spine that account for displacement of the patient's spine as it is being displaced or deformed by the robotic action. Further, the end point can be where the displacement will be applied.

In certain embodiments, a compact in situ fluoro-CT can be used to perform imaging of patient's spine during the surgical process.

In still another aspect, there is provided herein a method for a conducting a minimally invasive surgery, comprising:

capturing one or more pre-operative images of a surgical site to create a stereoscopic model;

displaying the one or more captured pre-operative images of the surgical site on at least one display device at a surgeon console;

plotting a surgery using the captured images displayed at the surgeon console and specifying the general location of one or more tracking system attachment points;

placing the one or more tracking system attachment points at least adjacent to the surgical site;

capturing one or more intra-operative images of the surgical site and layering those images with the captured pre-operative images to create a working stereoscopic model of the surgery site;

switching to a master-slave mode in the surgeon console, where one or more input devices of the surgeon console are used to couple motion into minimally invasive surgical instruments in which the one or more input devices are used to interact with a graphical user interface;

overlaying the graphical user interface including an interactive graphical object onto the one or more working stereoscopic model of the surgery site displayed on the at least one display device at the surgeon console,

wherein the interactive graphical object is related to a physical object in the surgical site or a function thereof and is manipulated by the one or more input devices of the surgeon console; and

rendering a pointer within the one or more working stereoscopic model of the surgery site displayed on the at least one display device for user interactive control of the interactive graphical object,

wherein the master-slave pointer is manipulated in three dimensions within the one or more working stereoscopic model of the surgery site by at least one of the one or more input devices of the surgeon console.

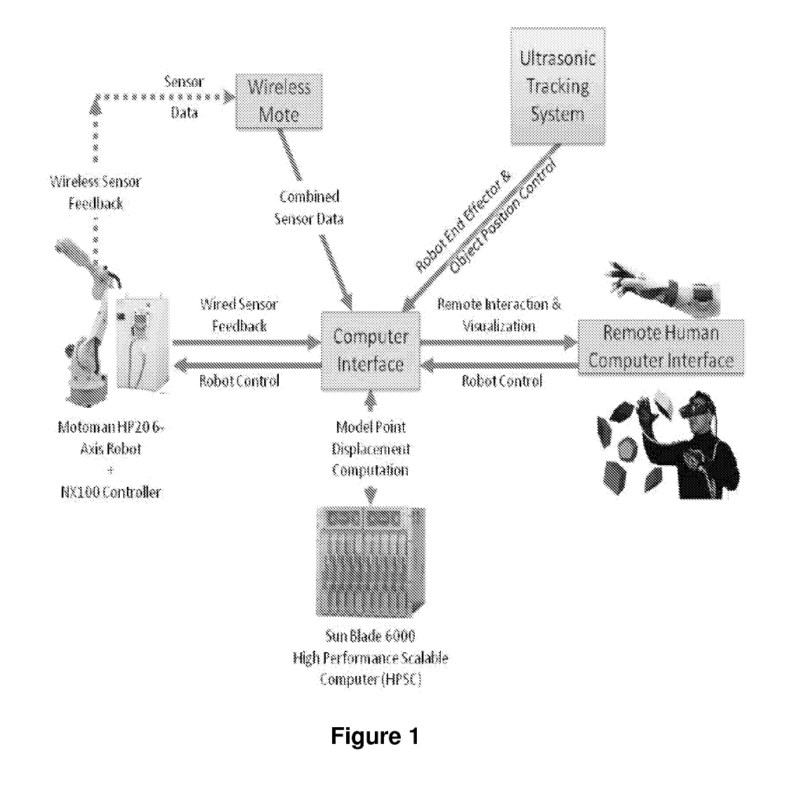

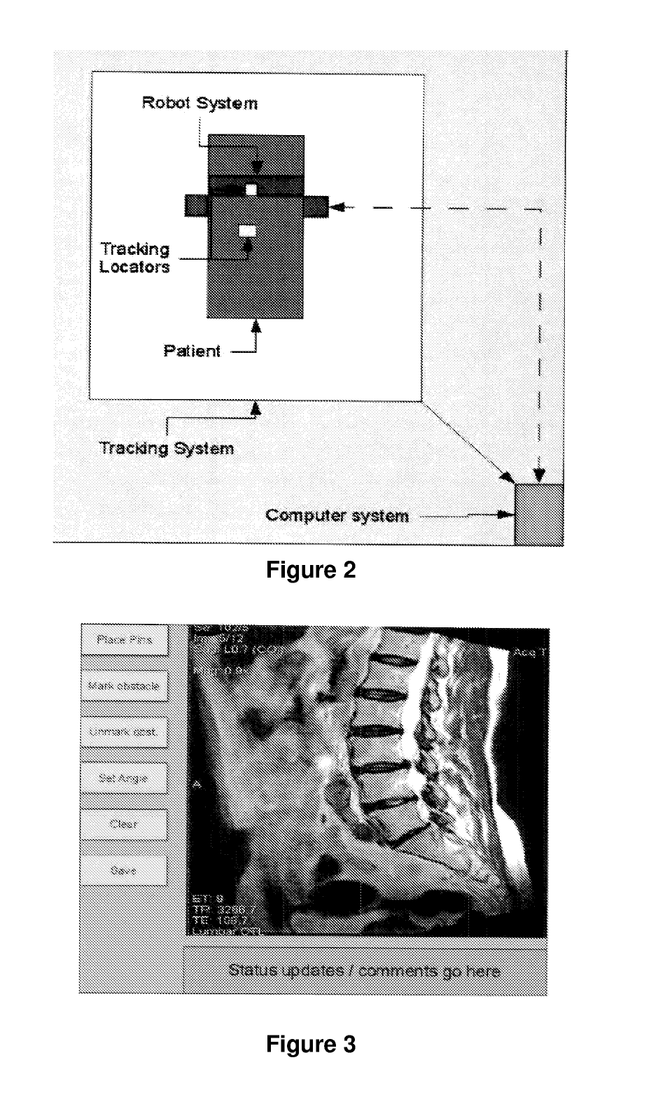

Referring first to the schematic illustrations of FIG. 1 and FIG. 2, there is shown a robotic minimally invasive surgery system (MISS) 10 that includes a processor or computer interface 12 that is in communication with a robotic system 14, a remote human computer interface 16 and a tracking system 20.

The robotic system 14 is configured to perform one or more desired functions. For example, the robotic system 10 can be mounted close to a surgical operating table and navigated using precise control signals wirelessly transmitted from a control station. It is to be understood that end-effectors specially designed to perform, for example, facet screw placement can be integrated with a robotic arm on the robotic system, along with one or more sensors that provide appropriate feedback signals. The computer 12 can be an advanced graphic processor that acts as a high performance computing platform for fast accurate real-time 3D spine modeling. Also, in certain embodiments, an ultrasonic tracking system can be employed to provide line-of-sight vision.

The robotic system 12 can be extended to perform all types of spinal surgical procedures including drilling, screwing and implant insertion. The robotic system 12 can be also adapted to perform other types of surgeries. Thus, the minimally invasive robotic surgery system 10 described herein can be useful to reduce patient trauma and cost of surgery, and to minimize radiation exposure to the personnel present in the surgical operating room.

In certain embodiments, the 3-D finite element (FE) modeling of complex objects can be performed in real-time as the object is being deformed or displaced. The position and orientation of the object can be sensed and tracked using a high-performance sensing environment. The 3-D modeling data generated can be transmitted over a wide bandwidth network with minimum latency. When deployed in a closed-loop configuration, this information, in turn, can be used to precisely control the movement of a robot operating on the object.

The tracking system 20 includes one or more tracking locators 22 that are placed at specific predetermined locations and one or more embedded sensors 22 (as shown in FIG. 2). It is to be understood that the tracking system 20 can also include suitable tracking hardware, wired and/or wireless sensing features and suitable communication features that enable the tracking system to be collect and send data to the computer 12.

Although described as a “processor” or “computer,” the computer 12 may be a component of a computer system or any other software or hardware that is capable of performing the functions herein. Moreover, as described above, functions and features of the computer 12 may be distributed over several devices or software components. Thus, the computer 12 shown in the drawings is for the convenience of discussion, and it may be replaced by a controller, or its functions may be provided by one or more components. For example, in one embodiment, the computer 12 can include a High-Performance Scalable Computer (HPSC) infrastructure for real-time spine modeling, and advanced human-computer interfaces.

The computer 12 connects the robotic system 14, the remote human computer interface 16 and the tracking system 20, all in a closed-loop configuration with feedback and control mechanisms for intelligent maneuvering of the robotic system 14. The robotic system 14 can be integrated with any suitable end-of-arm tooling package. In certain embodiments, the robotic system 14 is configured to be placed close to the subject. In certain embodiments, the tracking system 18 can be an Intersense IS-900® ultrasonic/inertial tracking system, which provides imaging of the subject without the line-of-sight restriction.

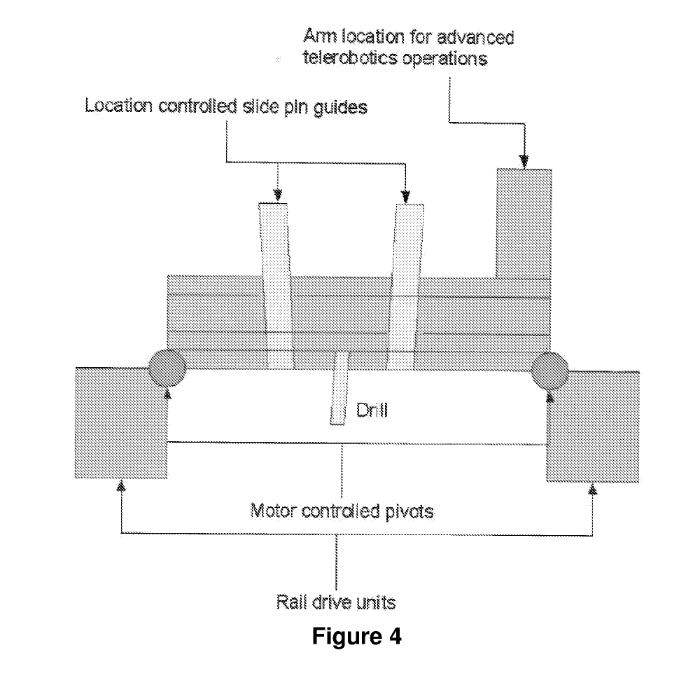

According to one embodiment, a series of images are taken either pre- or peri-operatively and are combined to form a first 3D model, as generally illustrated in FIG. 3. The first 3D model can then be transferred to a software program that is either integral with processor 12, or separate from the processor. FIG. 4 is a schematic illustration of a tracking system.

Using this software, the surgeon can layout points of interest to the particular procedure, including, for example, in a spinal fusion surgery, specifying the spinal pin insertion locations and angles to be used, any objects in that area that must be avoided, and the general location of one or more sensors of the tracking system.

For example, in a spinal fusion surgery, once in surgery, the surgeon can place one or more of the sensors on a bony appendage of the spine that near the surgical working area that had been specified in the software. Once the sensors are placed, one more set of images can be taken to create another 3D model and to locate the placed tracking sensor. The software then can combine the new 3-D model with the first 3-D model to create a working 3-D model.

As the patient moves in surgery, due to any number of factors (for example, patient breathing, forces applied by the surgeon, forces from the robotic system, and the like) the tracking system 18 receives data from the sensors, communicates such data to the processor 12, where the processor updates the working 3-D model. At this point, the robotic system 14 can be initiated to begin the surgical procedure.

Multiple stages of the surgery can then be executed. The computer 12 can use the working 3-D model in conjunction with data on the robot location and the planned surgery, and provide a set of instruction to the robot. For example, in one non-limiting embodiment, the computer could instruct the robot to move into and maintain position, and provide a method for allowing the surgeon to complete pin insertion while guided by the robot (ex. pin guide system).

In another non-limiting embodiment, the computer can instruct the robot to move into and maintain position, and then further instruct the robot to insert the pins on its own.

In another non-limiting embodiment, the computer could allow for telerobotic control through virtual reality. In this embodiment, the robot can be outfitted with one or more imaging systems (e.g., cameras, ultrasound, and/or the like) to provide a real-time image.

In certain embodiments, the robotic system 14 can also be fitted with one or more devices that can simulate a human hand, and/or provide feedback on such parameters as, for example, pressure, torque, strain. This information can be collected by the computer 12. A surgeon can then log into a virtual reality system. For example, the virtual reality system can include data gloves, a tracking system, one or more haptic feedback devices, and a stereo visualization system. The virtual reality system can be anywhere in the world, and the surgeon will be able to log into the computer and perform the surgery as if he were in the room.

In certain embodiments, the tracking system 20 can be included in or otherwise associated with the computer 12. The tracking system 20 provides information about the position of the working area, such as a particular vertebra during spinal surgery.

As the subject is displaced and/or deformed due to the robotic action, the tracking system 20 interfaces with the computer 12 to generate real-time update data (e.g., a 3D FE model) corresponding to the new position of the vertebra.

This updated data is then used to precisely control and move the robot to perform the next desired operation. The entire process is completed within a very short time so that robot movement and object displacement/deformation are nearly synchronized.

It is to be understood that, in calculating forces on and/or changes in position due to such force, a comparison is made between a kinematic (or calculated) position of the tool versus an actual position of the tool. Such comparison represents force on the vertebra. When forces applied to the robotic tool, such as a static force experienced when the robotic tool is pressing against an obstruction, this force can cause the vertebra to be moved slightly.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

Architecture of the System

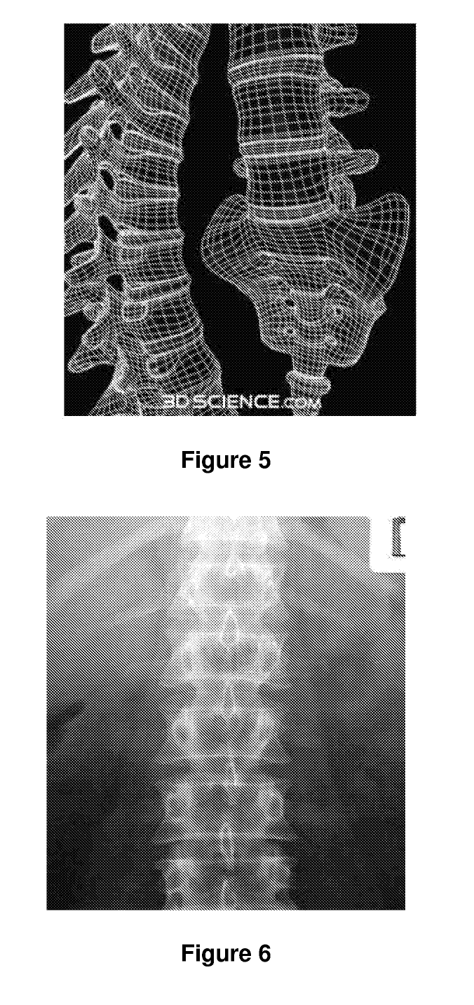

The implementation of the system described herein generally includes a number of factors working together. The first stage is to obtain pre-operations imaging. This is normally in the form of MRI or CT scans. Using this data, a 3D model is produced, as seen in FIG. 5 and FIG. 6.

Using data from the FE (finite element) model, a model for the patient specific FE model is created. It is from this model that the surgeon can then associate tracking points, and prepare for the surgery. Once in the operating room, the physical placement of the tracking point can take place.

At this point, additional 3-D imaging is done in order to accurately register the marker's physical location with their locations on the 3-D model. With the trackers registered, model updating can begin. Knowing the position of the tracked points, as well as their associated positions on the 3-D model, the surgical team can then compute the other points in the 3-D model using finite element analysis. This is done so that, when the robot is moving, the robot needs to know exactly where its source and its target are, as well as anything between that should affect its trajectory. As the path changes, the robot can be re-routed in real time.

At the center of this architecture is a wireless sensor mote. This allows a great deal of flexibility, specifically in expansion of the system. For example, in embodiments where the robot is outfitted with a blood sensor on its tip that indicated excessive pooling of blood, the mote is able to make the decision to send the necessary commands to the robot to correct the problem. Because the control is handled in the same module that is polling the sensors, reliability is increased, along with faster response times. An additional benefit is that the entire system becomes wireless—thus it is possible to take manual control of the system at any time from any number of devices.

Tracking System

While optical tracking is the traditional approach in the operating room, the requirement of line of site vision at all times is, however, not an acceptable limitation for MI surgeries.

In certain embodiments, the system described herein can use an ultrasonic/inertial tracking system, such as the Intersense IS-900 ®, in order to overcome these limitations. In other applications, where the change between movements is larger, an ultrasonic/inertial tracking systems other than the IS-900® device may be suitable. Still, since the motion realized during a minimally invasive (MI) surgery does not rely on high velocities or accelerations, the accuracy of the optical system can be improved without the previously encountered “line of sight” restrictions.

In certain types of surgeries, however, the motions that are required in this type of situation are motions such as patient respirations, working forces from the robot, and any corrections that the surgeon makes during the surgery.

Tracker Results

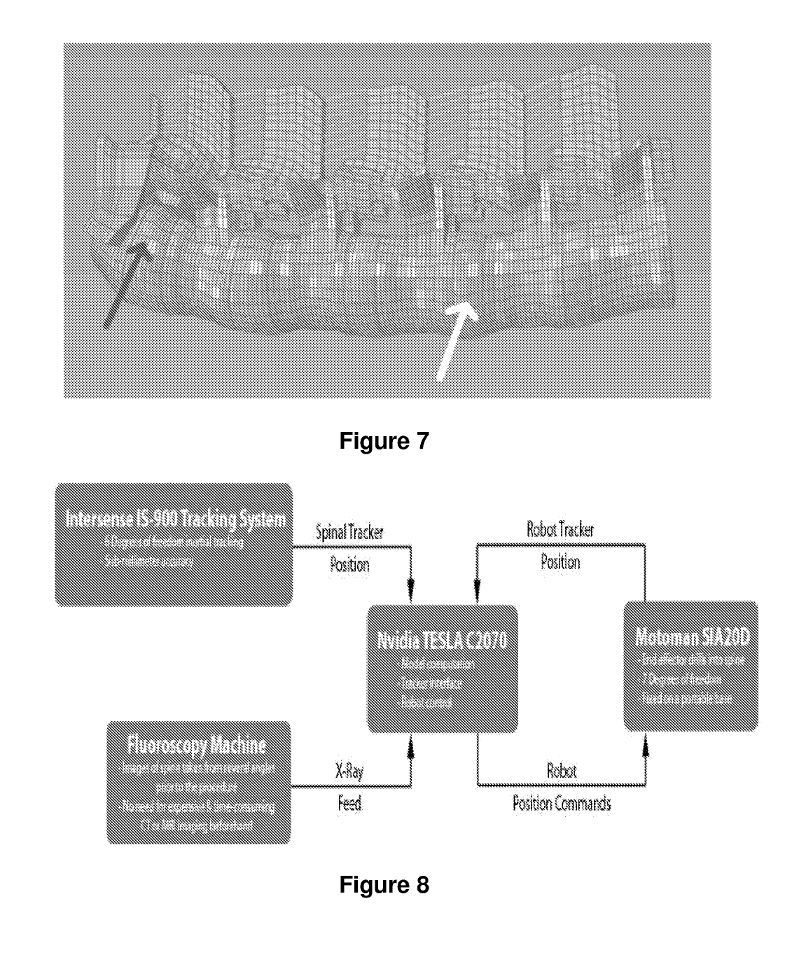

Tests were performed using a spine analog that is constructed of fiberglass, foam and rubber. The spine was tested at intervals of 1, 2, and 3 cm in X, -X, and Z directions. FIG. 7 is a 3-D model of a spine, indicating where loads were applied and measured.

FIG. 8 is s schematic illustration of another example, where the minimally invasive robotic surgery system comprises wireless and human-computer interfaces, real-time 3D FE model reconstruction of the displaced spine that can be patient-specific and can provide accurate feedback, a robot that is equipped with end effectors, a GPU processor and a small robot capable of reduced payload. Additionally, the robot can be mounted close to a surgical operating table and a compact in situ fluoro-CT capability can be integrated to perform imaging of patient's spine during the surgical process.

This type of minimally invasive robotic surgery system can be seen in FIG. 8, where the system includes a Motoman SIA20D® robot, an Intersense IS-900® ultrasonic tracking system, an Nvidia TESLA S2070® GPU computing system, and a fluoroscopy machine. The Motoman SIA20D® robot is a robust, 7 degrees of freedom (DOF) manipulator with a modular end effector system. With the 7 axes and large reach area, the robot can move to any position at any angle on the operating table. For portability, the robot and its controller can be mounted on a wheeled base.

It is to be noted that any type of fluoroscopy machine can be used, as long as it is powerful enough to penetrate and generate accurate images of patient's pelvic spine area. The robot can be mounted on a sturdy portable base close to a mobile surgical operating table. The table is accompanied by a cart that houses the computing and controlling hardware.

End effectors for virtually any type of surgery can be designed and integrated with the robot. The SIA20D® robot also has ±0.1 mm repeatability accuracy, which rivals any human holding a scalpel or any other surgical tool. For example, a custom end effector can be used perform the facet screw placement of X-spines FDA Class II certified Fixcet® system. Also, the end effector designs can be modular. Hence, changing the tools is very fast and easier.

The minimally invasive robotic surgery system configuration employs two types of trackers, a first tracker type built into the end effector(s), and a second tracker type comprising a wireless tracker that can be removably mounted onto the vertebrae of patient's spine. In the example herein, the wireless tracker measures ˜10 cm*1 cm*1 cm, and can be rigidly mounted on the vertebrae using a clip. The tracking system's latency is on an average below 20 ms (considering I/O latency as well) and has an average tracker accuracy of ±0.5 mm. The trackers offer 6 DOF, i.e., the trackers read not only their x, y and z position coordinates under the tracking grid, but with the help of accelerometers and gyroscopes, they can also transmit the angle they are at. More than one tracker on the spine can be used to achieve higher accuracy if needed. The advantage of using an ultrasonic tracking system is that it offers line-of-sight vision. Even if the robot is in the way of the tracking system during a surgical action, accurate tracking of the spine can be accomplished.

The computing system communicates with the components of the system, can receive and process x-ray images from the fluoroscopy machine, the tracker positions from the tracking system, and can control the robot. The computing system can provide high-performance computing (HPC) due to its use of GPUs. For example, one S2070® computing system four Nvidia Fermi class GPUs, each with 448 stream processor cores, which is a total of 1792 stream processor cores, delivering 2 Teraflops double precision floating point operation performance and 4.13 Teraflops single precision floating point performance. This kind of floating point operation performance is optimal for finite element analysis (FEA), and for the ABAQUS application, an FEA software suite, to compute any changes in the position and orientation of the spine during the surgical procedure.

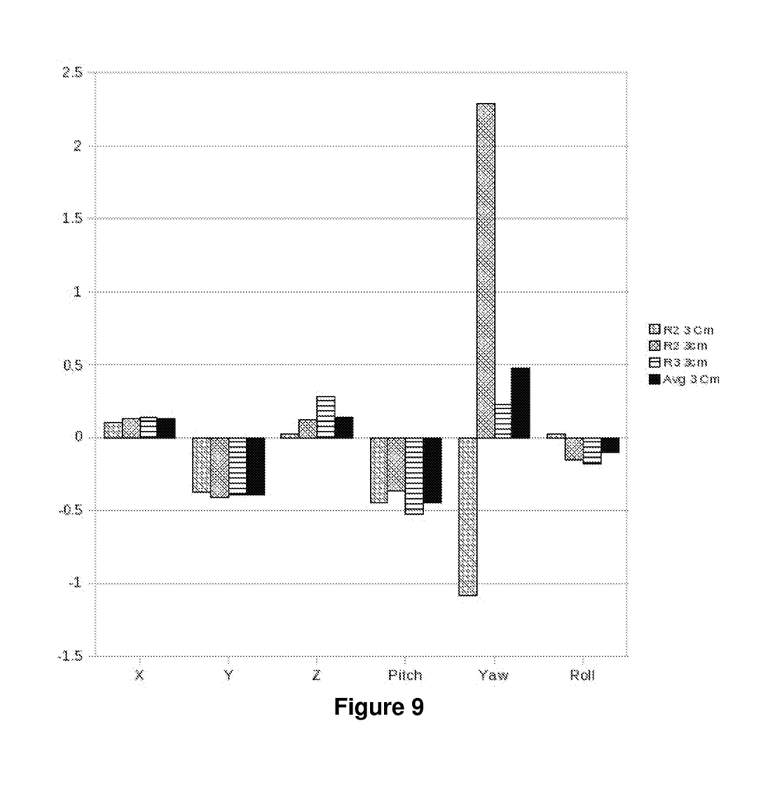

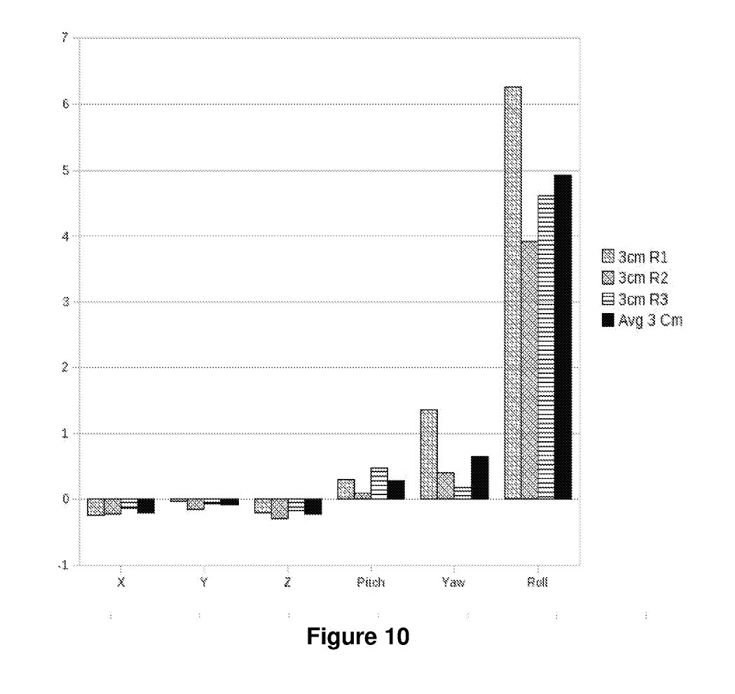

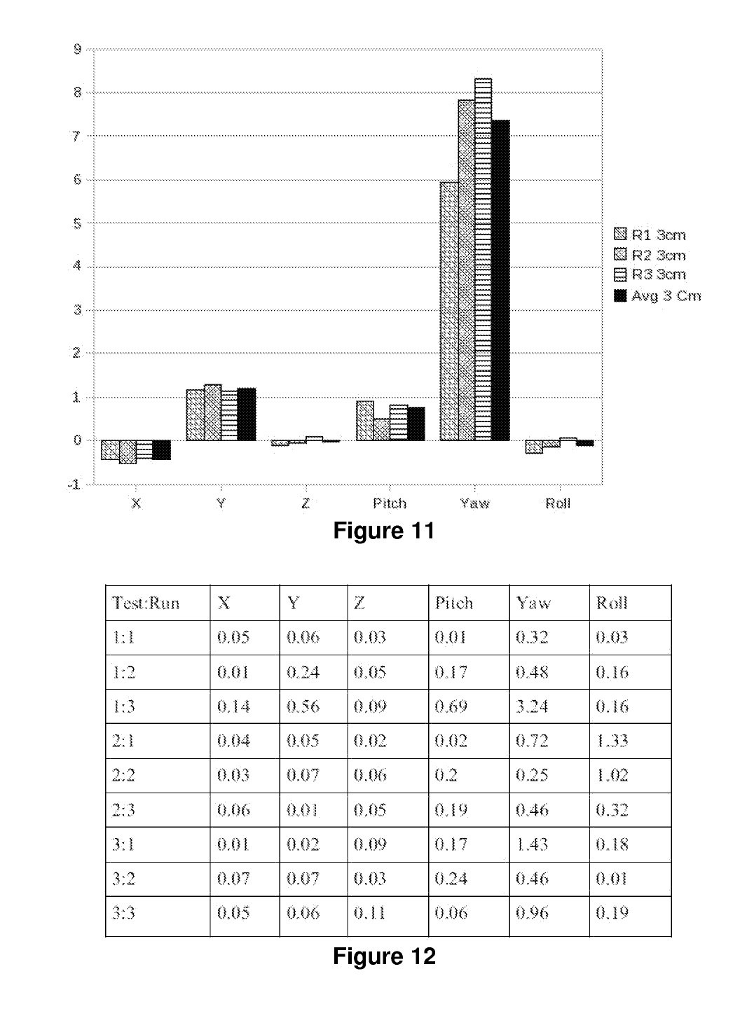

FIG. 9, FIG. 10 and FIG. 11 show the measurement results from the displacements applied in the test directions on the spine 3-D model. As can be seen from the graphs in FIGS. 9-11, the X, Y and Z coordinates maintain an acceptable precision. FIG. 12 shows the standard deviation data for the test runs.

Computations

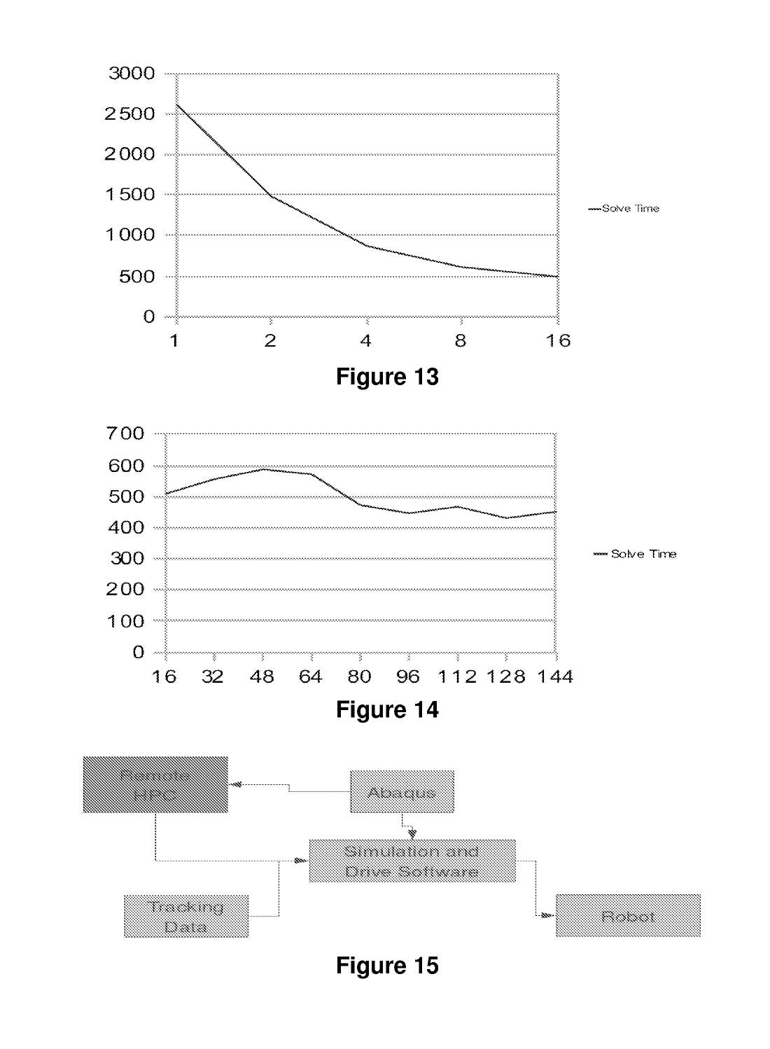

FIG. 13 and FIG. 14 illustrate the computation time required for convergence of the FE 3-D model system described herein. As can be seen, the system is able to drastically reduce computation time. For example, while running in threads mode, the computations can be done on an on-site computer. In certain embodiments, one or more steps can be taken to decrease computation time. While the model used approximately 170,000 nodes, many of the nodes could be coupled in embodiments, where there is not a concern with inter-bone torques and loads. In other applications, this can be reduced into the 10 s of thousands of nodes.

Other steps can include, for example, using MPI which offers multiple parameters to tweak in an attempt to find the optimal settings. Additionally, performance gains can be found through use of a core processor which achieve ˜1 TFlop on a single die, compared to ˜90 Gflop.

Operation of Robotic Surgical System with Advanced Electronic Tracking

The system described herein provides for certainty of the location of all points of interest throughout a surgical procedure which ensures accurate placement of surgical implant. This accurate placement, in turn, can then decrease the radiation exposure to both the patient and the surgeon. One operational procedure of the system is detailed in FIG. 15. Tracking data is obtained by the software daemon. The daemon then modifies the FE model to account for the change in position realized by the tracking setup, and is sent to the HPC for computation. Upon completion, the offset that has been calculated for the point in question is passed back to the daemon, and thusly on to the robot with any additionally needed offsets. This allows the robot to keep its position up to date relative to the point of interest.

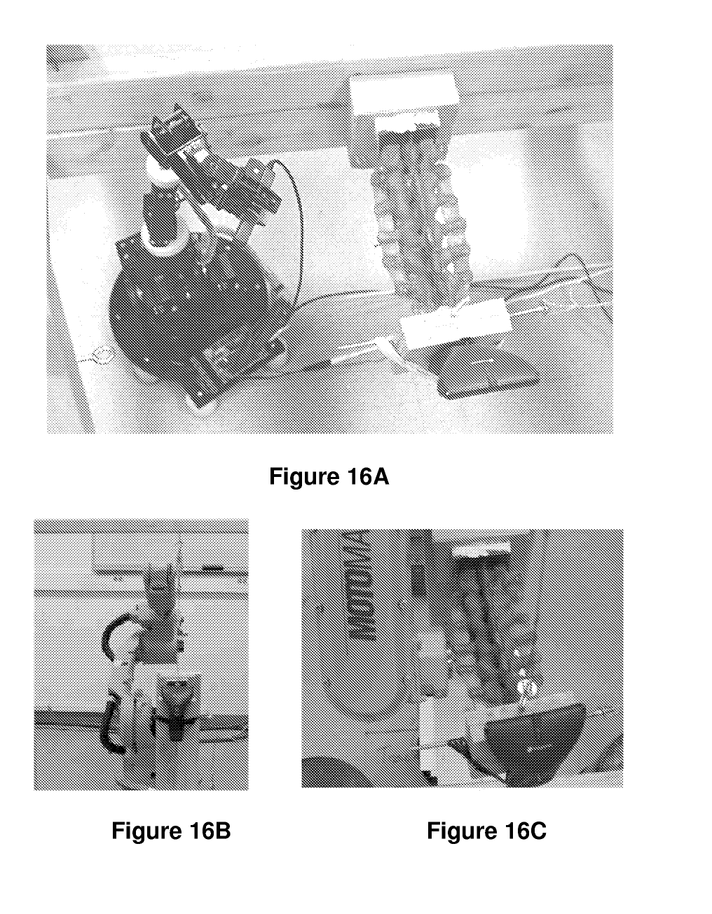

The test setup is as can be seen in FIGS. 16A-16C. The spine is mounted horizontally supported by S1. A nail is inserted transversely in a position that corresponds with the MIDPOINT node in the FE model. This will be the node whose position will be calculated.

The robotic arm used in this example was a Cyton 7A ® from Energid, which is a 7 DOF general purpose robotic arm and can have libraries to calculate kinematics, as well as to control the robots movements.

One of the trackers is mounted at ENDPOINT, the point at the load end of the spine, and the other is mounted to the robot. The static offsets due to the mounting of these trackers is accounted for in the software. The end point is where the displacement will be applied. The displacement is applied via the turnbuckles located around the testing rig. These turnbuckles are placed in such a fashion that they are directly positioned with the midpoint of the block attached on the end point in an at rest position. This allows the displacements to be applied as linearly as possible.

To perform this test, the inventors first recorded the initial points, EP0 and MP0, for ENDPOINT and MIDPOINT. Subsequently, a 2 cm lateral offset was applied to the spine. This was performed by adjusting the turnbuckle until the endpoint tracker reads the proper displacement. The new positions, EP1 and MP1 were recorded. At this point, the offsets for EP1 were fed to the FE model solver, which returned a value for MP1. At this point, the robot was then be moved into its relative position—in this case, directly to the calculated position.

Results

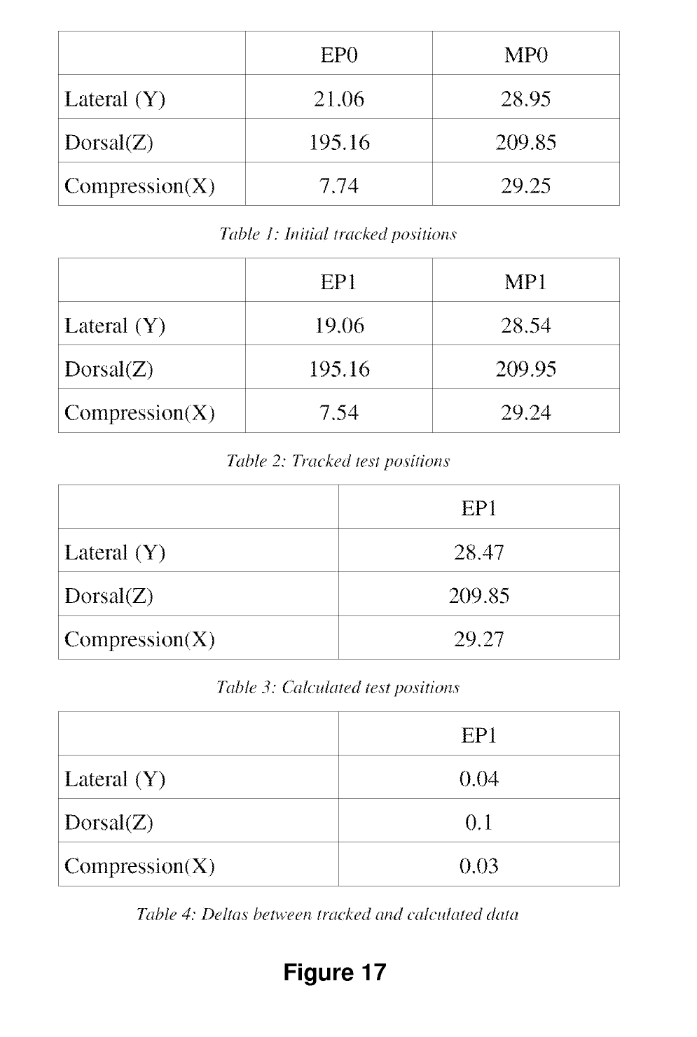

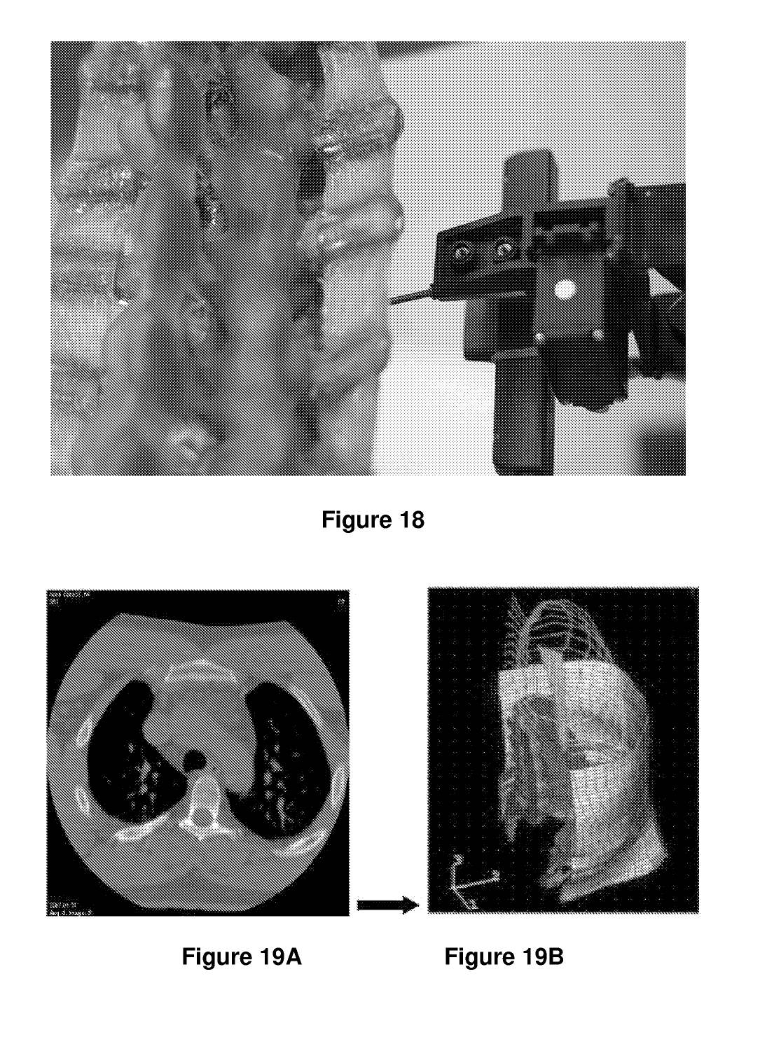

As can be seen from the data in FIG. 17, showing Tables 1-4, as well as the robot position in FIG. 18, the calculated values are quite accurate. The solve time for the 3-D model was approximately 90 seconds. It is to be noted that, in other embodiments, minimizing the solve time may include using a rigid body model as opposed to the FE model.

By integrating with surgical planning software, the system described herein can also allow for tracking of more specific offsets. For example, if the goal of the robot is to hold instruments in place for a surgeon while maintaining a relative position to a specific point, then the integration of the planning software will allow for a more accurate description of where exactly the robot should be given the points position.

Spinal Example

The implementation of the minimally invasive robotic system uses a 3-D FE model of the spine in order to track precise robotic movements. FIG. 19A and FIG. 19B illustrate 3D FE models generated using ABAQUS software. The models were adapted for the spine analog shown in FIGS. 16A-16C. For precise robotic movements associated with probing, drilling, screwing and insertion operations present in a typical spinal surgical procedure, an accurate patient-specific 3D FE model of patient's spine is needed at the beginning of the surgical procedure.

To accomplish this objective muscle origin, physiological cross-sectional area and insertion site information, the 3D line of action, and the transverse cross-sections of the vertebral bodies from T12-S1, using very low dose CT and OpenMRI scans can be gathered.

First, transverse scans can be obtained, from T12 to S1, and the lateral view of the spine for each subject lying supine using the CT scanner. The CT images can be digitized to delineate the outlines of various soft and hard tissue structures, like the facets and pedicles. Muscles that can be indentified include the abdominal group, psoas, quadratus, and muscles of the back. The trunk width and depth and the distance from the back to the pedicle can also be identified. The outlines of various structures can be digitized in order to account for intra-observer errors. These data can be used to develop a patient-specific 3D FE model, as described below.

The 3D FE model of the ligamentous spine can be created using 3D MRI and CT images. The images can be imported into the MIMICS® software to generate a 3D volume-rendered geometric model of the bony structures. First, each CT image can be segmented to delineate bony regions. Secondly, the bony regions that belong to the same structural component (e.g., a vertebra) can be merged in MIMICS. A 3D volume-rendered geometric model can then be exported as an IGES file. TrueGrid® softwar can be used to import this IGES file and generate a 3D hexahedral FE mesh in ABAQUS® format.

In this 3-D FE model, the vertebral body can be modeled as a hard cortical shell which envelopes the soft cancellous bone. The posterior bony elements can be joined to the vertebral body posterolaterally. The disc annulus can be modeled as a composite of a solid matrix (hexagonal elements) with embedded fibers (using the REBAR parameter) in concentric rings around nucleus. Fluid elements which allow for hydrostatic pressure can be used to define nucleus. All seven major spinal ligaments can be represented. Naturally changing ligament stiffness (initially low stiffness at low strains followed by increasing stiffness at higher strains) can be simulated through the “hypoelastic” material designation, which allows the definition of the axial stiffness as a function of axial strain.

Three-dimensional two-node beam elements can be used to construct the ligaments. The apophyseal (facet) joints can be defined with non-linear surface-to-surface contact definition. The “softened contact” parameter with non-linear stiffness can be used to simulate the cartilaginous layer between the facet surfaces.

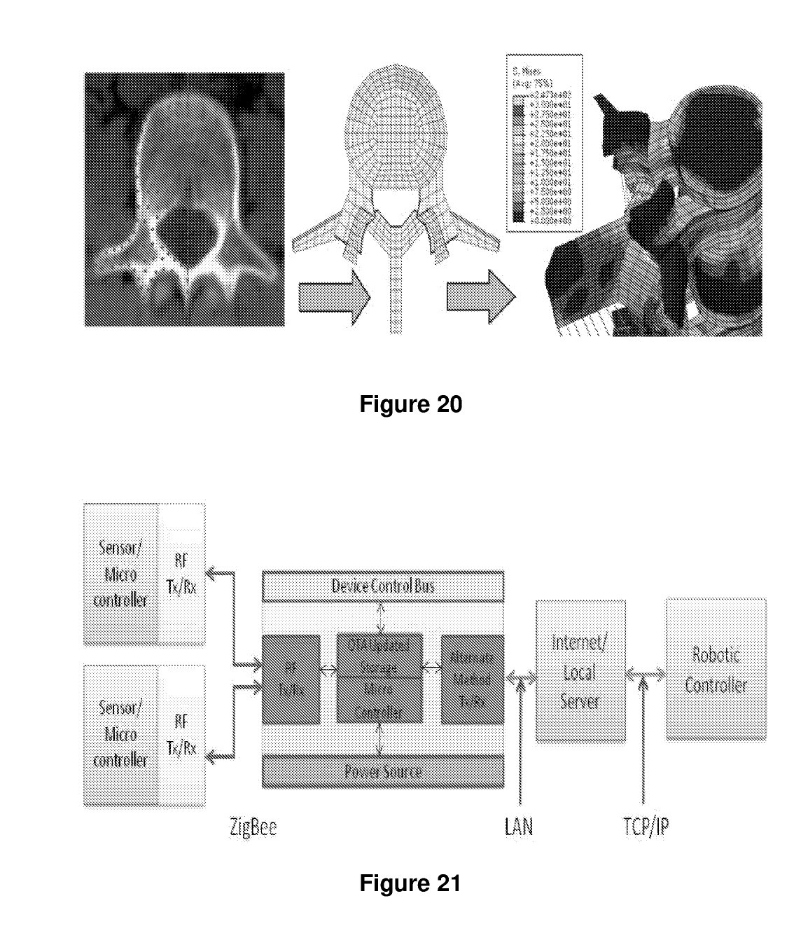

An initial clearance as determined from the CT/MRI scans can also be simulated. FIG. 20 shows the process flow of generating a 3D FE spine model from CT scans. Using the FE model approach can allow the use of the external markers at only one vertebral body and compute parameters of interest at other levels even if the vertebral bodies move, like due to breathing.

Using ABAQUS, a minimum 3D FE spine model computation time of 90 seconds or less can be obtained. To decrease computation times, the spine 3D FE model can be simplified, more compute nodes can be added, compute nodes can be doubled, a better networking interface, such as quad data rate (QDR) 12 speed Infiniband networking, can be used and model data can be generated prior to surgery by mapping out the area in which the robot is likely to move in. Once the data are easily accessible in the HPSC system memory and as soon as the tracking system senses displacement within the estimated area, the displacement of the point from the pre-computed models can be extrapolated in the system memory. The system memory of each node is accessible from the others, by the method of Remote Direct Memory Access (RDMA) and an Infiniband connection. Additionally, with improved processor speeds being released by Intel and other such companies, model computation times of a few milli-seconds can be achieved.

In the minimally invasive robotic system shown in FIG. 8, the 3D FE model of the spine is accurate at all times and the robot knows the exact location and position of a patient's spine. When the robot operates on a patient, a patient's spine may be deformed and/or displaced due to applied force and torque. Consequently, the model is continuously updated in real-time so that the robot can be guided to perform appropriate tasks without any time delay. Markers can be placed on the spine at predetermined locations and trackers can be used to register these markers at identical locations in the 3D FE model of the spine. By properly tracking these markers as the spine is being deformed and/or displaced by the robotic action, the 3D FE model can be updated in real-time and the information used to navigate the robot. To obtain the desired accuracy, the number and location of markers can be properly chosen. For example, accurate placement of the anchors within a given location, particularly in the spinal vertebra can require the margin of error of a functional robotic arm to be within 1 mm.

The 3D position of a given tool in reference to preselected markers can be tracked by utilizing optical scanners. Such markers can be placed on the spinous process with the help of a clamp or in the posterior superior iliac spine. The pedicle of the vertebra can also be used as an alternate marker. By using the pedicle as a marker, the need for a separate incision to place a marker will be unnecessary. The facet joint of the level being operated upon can also be used as a marker for guidance of the robotic arm.

Using ultrasonic/inertial tracking systems, such as the Intersense IS-900, can give a surgeon line-of-sight vision at all times. Motions that are typical in a minimally invasive spinal surgical procedure deal mostly with respirations, working forces from the robot, and corrections that the surgeon may make during the surgery.

Prior to surgery, an appropriate number of markers and their locations on the spine can be determined. Initially, the model of the T12-S1 segment with bony and soft tissues details can be transferred to a patient lying supine on the operating table using three bony marks identified by the surgeon via the patient's vertebral CT scans. At that point, additional imaging can take place (if needed) to accurately register the physical location of markers with their corresponding locations on the 3D FE model. With the trackers registered, model updating can begin. By knowing the position of traced points on the patient, as well as their associated positions on the 3D FE model, other points in the model can be computed using the FE analysis. The surgeon then can define trajectories for the screw placement on the 3D FE model (which will have all the details). Also, under robot control, the surgeon can tap/drill pedicle screw holes for the placement of the instrumentation either by the robot itself or the surgeon. Furthermore, using the detailed FE model, the surgeon can compute the displacements/deformations that are likely to occur due to the force applied by the surgeon in tapping/drilling holes, etc. For this purpose, appropriate end effectors can be used to perform facet screw placement.

The patient-specific 3D FE spine model can provide the necessary spatial material information to determine the exact amount of force and torque to be used by the robot. For example, the robot can be equipped with wired sensors that can be used to provide the haptic, force and torque feedbacks as needed.

Example with Wireless Interface

In another example the minimally invasive robotic surgery system can include a wireless interface, as shown in FIG. 21. The wireless interface can communicate with the main robot controller and also separately with any of the sensors embedded in the robot. Because the mote has the capability to handle up to, and more than, 65,535 sensor nodes, this system architecture offers tremendous flexibility and scalability. A single mote can serve as a base station to control a sea of robots operating in an unstructured and unfamiliar environment. Because the mote can directly communicate with individual sensors as well as the central robot control unit, it provides a higher layer of authority. This feature is particularly attractive for independent control of a set of robotic functions from a remote location.

The mote can also be used to monitor and control other activities like vibrations and irregularities in performing a particular activity. The mote can also directly communicate with miniaturized sensors mounted on end effectors (such as vision and blood sensing) to provide added flexibility and simultaneous real-time control of a number of robots. The sensors can directly communicate with the mote which has the capacity to make decisions and wirelessly communicate with the robotic controller. For example, this improved system architecture can be ideal in situations where a number of robots need to be controlled from a remote location for training where the instructor has the master control. Thus the wireless mote can offer a great deal of flexibility, specifically in the expansion of the system and to enhance the functionality of the robotic activity. The entire system can become wireless, thus it is possible to take manual control of the system at any time from any number of devices.

This wireless interface can be different from traditional motes in a number of ways. It can be equipped with a pluggable communications interface and full OTA programmability and, when used in conjunction with the wireless sensors, it can support up to, and more than, 65,535 sensors per mote. By allowing over the air programming, complexity in the mote design can be eliminated, providing two advantages: lower cost and reduced power consumption. Power consumption can be further reduced through the use of a least power transmission algorithm, wherein the mote in question broadcasts to the closest eligible receiver, which is then forwarded on to the destination through the said mote in a similar fashion. By doing this, the power provided to the transceiver can be adjusted to use only the amount needed for that individual broadcast. Since the data is broadcast wirelessly directly from the sensor to the mote, the data is encrypted at the sensor level. Due to the relatively small amount of data that is transmitted between sensor and mote, the RSA algorithm can be used to encrypt the data. Using a modulus of 2048, a 256 byte message can be sent using RSA. By using the Atmel AT97SC3203 chip, processing time can be reduced to just 100 milliseconds for a 1024 bit modulus, or 500 milliseconds for a 2048 bit modulus.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A system for conducting minimally invasive surgery, comprising:
a three-dimensional (3-D) finite element (FE) model of a surgical working area that can be updatable in substantially real-time;
one or more markers operable to be placed at the surgical working area in at predetermined locations, the trackers being operable to register locations of the markers at identical locations in the 3-D model;
a robotic system operable to know the exact location and position of surgical working area; and
a software program operable to: i) track the location of the markers as the surgical working area is being deformed and/or displaced by action of the robotic system; and ii) update the 3-D model so that the robot can be guided to perform one or more tasks at the surgical working area without any substantial time delay.

2. The system of claim 2, wherein, the software program is operable to compute the displacements/deformations that are likely to occur due to the force applied the actions of the robotic system.

3. A system for conducting minimally invasive surgery comprising:
i) obtaining a three-dimensional (3-D) finite element (FE) computer model of a surgical working area on a patient;
ii) determining at least one of a position and orientation of a surgical tool that is positioned and oriented within an image plane defined by the 3-D model; and
iii) modifying at least one of the position and orientation of the 3-D model with respect to the image of the tool in the image plane such that the 3-D model approximately overlays the image of the tool so as to generate a corrected position and orientation of the tool; and
iv) tracking the tool by processing tool state information from step iii) using ultrasound coupled with the 3-D model.

4. The system of claim 3, wherein the tool state information is continuously provided at a sampling rate for processing.

5. The system of claim 3, wherein the signal emanates from the tool.

6. The system of claim 3, wherein the signal reflects off of the tool.

7. The system of claim 3, wherein the determination of the tool position and orientation comprises:
determining one or more estimated positions and orientations of the tool relative to a fixed reference frame from the sensor information;
determining one or more estimated positions and orientations of the tool relative to an ultrasound reference frame from the image information;
translating the one or more estimated positions and orientations of the tool from the fixed reference frame to the ultrasound reference frame; and
processing the one or more estimated positions and orientations to generate the tool position and orientation relative to the ultrasound reference frame.

8. The method according to claim 7, wherein the one or more estimated positions and orientations derive from time sampled information provided by one or more sensors coupled to a mechanism for manipulating the tool through the incision in the body, and the one or more ultrasound estimated positions and orientations derive from sampled ultrasounds provided by one or more ultrasound devices so as to capture locations of the tool.

9. The method according to claim 8, wherein one or more measures are derived for the one or more estimated positions and orientations.

10. The method according to claim 8, wherein the measure for the one or more estimated positions and orientations is determined from a difference between one of the estimated positions and a position being commanded by a command signal controlling the mechanism for manipulating the tool.

11. The method according to claim 10, wherein the determination of the tool position and orientation includes processing the ultrasound information to identify a marker on the tool, and determine an orientation of the tool using the marker.

12. The method according to claim 11, wherein the determination of the tool position and orientation includes:
generating a computer model of the tool using the ultrasound sensor information so as to be positioned and oriented within a plane defined in the ultrasound information, and
modifying the position and orientation of the computer model with respect to an image of the tool in the image plane until the computer model substantially overlays the image.

13. A minimally invasive robotic surgery system, comprising:
one or more ultrasound devices operable to provide data from which tool state information is generated when a tool is inserted and robotically manipulated through an incision in a body; and
a processor operable to process the non-endoscopically and endoscopically derived tool state information for tracking the state of the tool.

14. The system of 13, further comprising a mechanism used for manipulating the tool through the incision in the body, wherein the one or more ultrasound devices include one or more sensors providing sensor data representing tool movement information according to such manipulation.

15. The system of claim 14, wherein the sensor data includes digitized samples of an identifiable signal emanating from or reflecting off the tool so as to indicate the position of the tool.

16. The system of claim 13, wherein the processor is further operable to identify a marker on the tool, and to determine an orientation of the tool using the marker while tracking the state of the tool.

17. The system of claim 13, further comprising a mechanism used for manipulating the tool through the incision in the body, wherein the sensor data represents kinematic information according to such manipulation.

18. The system of claim 13, wherein the processor is operable to generate a 3-D computer model of the tool positioned and oriented within an image plane defined in the ultrasound captured data, and modify the position and orientation of the 3-D computer model with respect to an image of the tool in the image plane until the 3-D computer model substantially overlaps the image.

19. The system of claim 18, wherein the modification of the estimated position and orientation of the 3-D computer model with respect to the ultrasonic data of the tool in the captured image, comprises:
determining the modified position and orientation of the computer model that approximately overlays the tool image by minimizing a difference between the computer model and the ultrasonic data of the tool.

20. A tool tracking method comprising:
generating a plurality of estimated tool states for each point in a plurality of points in time, while the tool is inserted and being manipulated through an incision in a body; and
determining an optimal estimated tool state for each point in the plurality of points in time by processing the plurality of estimated tool states using ultrasonic techniques.
wherein the plurality of estimated tool states include an estimated tool state determined using only sensor data associated with a robotic mechanism for manipulating the tool, so as to be indicative of movement of the robotic mechanism.

21. The method of claim 20, wherein the plurality of estimated tool states includes an estimated tool state determined using only sensor data associated with the tool, so as to be indicative of a position of the tool.

22. A method of claim 21, wherein the plurality of estimated tool states include an estimated tool state determined using only ultrasound data generated by an external ultrasound device positioned so as to detect a tool inserted into and being manipulated through a incision in the body.

23. A minimally invasive surgical robotic system, comprising:
a tracking system for a robotic system operable to send signals;
a computer interface operable to receive the sent signals from the tracking system and to combine the sent signals with a three-dimensional (3-D) finite element (FE) computer model to provide sensor data;
the computer interface operable to transmit the sensor data to the robotic system; and
the computer interface operable to provide a closed loop system operable to transmit/receive sensing and feedback signals from the tracking system as a surgery is being performed;
wherein a real-time computer modeling is provided during surgery; the real-time computer modeling comprising an updatable three-dimensional (3D) finite element (FE) modeling of a surgical work area as such surgical work area is being displaced or deformed by the robotic action.

24. The minimally invasive surgical robotic system of claim 23 wherein the minimally invasive surgical robotic system navigates using precise control signals wirelessly transmitted from a control station.

25. The minimally invasive surgical robotic system of claim 23, wherein the minimally invasive surgical robot arm contains end-effectors and sensors that provide appropriate feedback signals

26. The minimally invasive surgical robotic system of claim 23, wherein the surgery being performed is any spinal surgical procedure including drilling, screwing and implant insertion

27. The minimally invasive surgical robotic system of claim 23, wherein the tracking system includes one or more reference points embedded at or near on the surgical working area and which appear in the three-dimensional (3D) finite element (FE) model of the surgery surgical working area.

28. The minimally invasive surgical robotic system of claim 23, wherein as the surgical working area is displaced and/or deformed due to the robotic action, the tracking system interfaces with the computer to generate a real-time update of the 3D FE model corresponding to the new position and shape of the object.

29. The minimally invasive surgical robotic system of claim 23, wherein the surgical working area is a patient's spine, and the three-dimensional (3D) finite element (FE) modeling of the patient's spine contains trackers placed at MIDPOINT (MP) nodes and ENDPOINT (EP) nodes in the spine that account for displacement of the patient's spine as it is being displaced or deformed by the robotic action.

30. The minimally invasive surgical robotic system of claim 29, wherein the end point is where the displacement will be applied.

31. The minimally invasive surgical robotic system of claim 29, wherein a compact in situ fluoro-CT is used to perform imaging of patient's spine during the surgical process.

32. A method for a conducting a minimally invasive surgery, comprising:
capturing one or more pre-operative images of a surgical site to create a stereoscopic model;
displaying the one or more captured pre-operative images of the surgical site on at least one display device at a surgeon console;
plotting a surgery using the captured images displayed at the surgeon console and specifying the general location of one or more tracking system attachment points;
placing the one or more tracking system attachment points at least adjacent to the surgical site;
capturing one or more intra-operative images of the surgical site and layering those images with the captured pre-operative images to create a working stereoscopic model of the surgery site;
switching to a master-slave mode in the surgeon console, where one or more input devices of the surgeon console are used to couple motion into minimally invasive surgical instruments in which the one or more input devices are used to interact with a graphical user interface;
overlaying the graphical user interface including an interactive graphical object onto the one or more working stereoscopic model of the surgery site displayed on the at least one display device at the surgeon console,
wherein the interactive graphical object is related to a physical object in the surgical site or a function thereof and is manipulated by the one or more input devices of the surgeon console; and
rendering a pointer within the one or more working stereoscopic model of the surgery site displayed on the at least one display device for user interactive control of the interactive graphical object,
wherein the master-slave pointer is manipulated in three dimensions within the one or more working stereoscopic model of the surgery site by at least one of the one or more input devices of the surgeon console.
